(19) United States
(12) Patent Application Publication
(10) Pub. No.: US 2017/0168074 A1
(43) Pub. Date: Jun. 15, 2017

(54) Method for diagnosing G-protein coupled receptor-related diseases

(71) Applicant: OntoChem GmbH, Halle/Saale (DE)

(72) Inventors: Robert RENNERT, Halle (DE); David KOSEL, Leipzig (DE); Lutz WEBER, Germering (DE)

(21) Appl. No.: 15/039,360

(22) PCT Filed: Sep. 30, 2014
(86) PCT No.: PCT/EP2014/070926

§ 371 Date: May 25, 2016

(30) Foreign Application Priority Data
Nov. 25, 2013 (EP) 13194260.9

(51) Int. Cl. G01N 33/74; G01N 33/574

(52) U.S. Cl. CPC G01N 33/74; G01N 33/57492; G01N 2333/726

(57) ABSTRACT

The present invention relates to a method for diagnosing a G-protein coupled receptor-related disease in one or more target cells, comprising: selecting a G-protein coupled receptor, the receptor being characterized in that it is: (i) differentially expressed in the target cells as compared to healthy control cells, wherein the expression level in the target cells is at least 10 times the expression level in the control cells; (ii) activated by a peptide ligand or a protein ligand; and (iii) upon activation by binding of a ligand efficiently internalized into the one or more target cells together the peptide ligand or protein ligand, wherein an internalization of at least 30% of the G-protein coupled receptor initially present in the cell membrane of the one or more target cells within less than 30 minutes after activation is indicative for the diagnosis of a G-protein coupled receptor-related disease.

### FIELD OF THE INVENTION

The present invention is directed to a method for the diagnosis of G-protein coupled receptor-related diseases by selecting a particular G-protein coupled receptor being activated by a peptide ligand or protein ligand and determining its internalization behavior.

### BACKGROUND OF THE INVENTION

Available methods for the biological testing and selection of peptide- or protein-drug-conjugates that are receptor ligands typically utilize an effective receptor related transport into cells expressing the selected receptor, selecting thereby molecules with disease targeting properties to enhance the selectivity and the therapeutic window of such peptide- or protein-drug-conjugates.

In many cases, however, the use of drugs as systemic treatments for diseases is hampered due to their small therapeutic window and insufficient selectivity against healthy cells. Thus, the design and the selection of drug molecules with disease targeting properties has become a main area of research and development. For example, one known strategy to increase the therapeutic window of highly potent cytotoxic drugs is to conjugate those molecules to cancer specific ligands such as antibodies (cf., inter alia patent publications US 2010/0092496 and US 2011/0166319), peptides (cf., inter alia patent publication US 2011/0166319) or small molecule ligands of receptors such as for example the folate receptor (cf., inter alia patent publications US 2011/0027274 and US 2011/0172254). In an alternative approach, drugs are conjugated to naturally occurring molecules that are internalized in vivo such as for example vitamins (cf. inter alia patent publication US 2010/0004276). Similarly, other than cytotoxic therapeutic principles may be utilized by such drug conjugates, for example a TNF protein-linked amino-peptidase N antagonist (cf. inter alia patent publication US 2011/0076234) has been described for the treatment of angiogenesis related diseases.

While general drug conjugating principles have been developed and are known, it is still not possible to predict which specific compound will be a useful therapeutic conjugate as a broad range of factors influence the potential therapeutic efficacy of such conjugates. In general, a useful drug conjugate must exhibit three major properties that are all desired for a selective and potent disease targeting effect: (i) selective targeting of disease target cells versus healthy control cells by binding to a specific disease marker; (ii) efficient and rapid internalization of the drug conjugate into the diseased cells; and (iii) release of the drug molecule, for example by cleavage from the conjugate within the cell, for example in lysosomes of the disease target cells. While selecting suitable disease markers has become a major result of genomic or proteomic profiling of diseases, the use of such markers for drug targeting is not obvious.

In addition, and given a suitable disease marker, it is often highly difficult to predict if the above desired properties are met by a specific drug-conjugate. For example, drug conjugation may significantly decrease both affinity and selectivity or other binding properties of the drug-conjugate towards its target. Also, internalization of the drug-conjugate may be a result of an unspecific transport into the cell that is unrelated to the disease such as for example a general endocytosis, decreasing thereby the therapeutic window. It has also been reported that internalization of for example antibody-drug-conjugates is slow or the re-cycling (the transport from the cell back into the extracellular space) is faster. In addition, the cleavage of the drug molecule from the targeting ligand may already happen in the extracellular space or in the blood, resulting in a toxicity of the conjugate. Alternatively, the cleavage within the cell maybe slower as required or results in a more inactive toxin by having a part of the linker still attached or may not happen at all.

Therefore, there is an ongoing need for methods that overcome the above limitations and that allow to efficiently select simultaneously receptors and useful drug conjugates that use these receptors for disease targeting.

Accordingly, it is an object of the present invention to provide such a method.

### SUMMARY OF THE INVENTION

In one aspect, the present invention relates to a method for diagnosing a G-protein coupled receptor-related disease in one or more target cells, comprising: selecting a G-protein coupled receptor, the receptor being characterized in that it is: (i) differentially expressed in the target cells as compared to healthy control cells, wherein the expression level in the target cells is at least 10 times the expression level in the control cells; (ii) activated by a peptide ligand or a protein ligand; and (iii) upon activation by binding of a ligand efficiently internalized into the one or more target cells together the peptide ligand or protein ligand, wherein an internalization of at least 30% of the G-protein coupled receptor initially present in the cell membrane of the one or more target cells within less than 30 minutes after activation is indicative for the diagnosis of a G-protein coupled receptor-related disease.

In preferred embodiments, the peptide ligand or protein ligand is conjugated to a drug molecule, and particularly wherein conjugation is accomplished by means of a cleavable linker moiety or a non-cleavable linker moiety.

In specific embodiments, the peptide ligand or protein ligand is a naturally occurring ligand of the G-protein coupled receptor. Preferably, the naturally occurring ligand is selected from the group consisting of cytokines, peptide hormones and neuropeptides, and particularly preferably selected from the group consisting of neuropeptide Y, peptide YY, pancreatic polypeptide, orexin A, orexin B, gastrin releasing peptide, bombensin, litorin, neuromedin B, neuromedin C, endothelin-1, endothelin-3, SDF-1, GROα, IL-8, melanocortin peptides, angiotensin II, bradykinin, cholestocytokinin, neuropeptide FF, and RFamide related peptides.

In other specific embodiments, the peptide ligand or protein ligand is an artificially modified ligand. Preferably, the artificially modified ligand is based on a naturally occurring ligand being selected from the group consisting of cytokines, peptide hormones and neuropeptides, and particularly preferably selected from the group consisting of neuropeptide Y, peptide YY, pancreatic polypeptide, orexin A, orexin B, gastrin releasing peptide, bombensin, litorin, neuromedin B, neuromedin C, endothelin-1, endothelin-3, SDF-1, GROα, IL-8, melanocortin peptides, angiotensin II, bradykinin, cholestocytokinin, neuropeptide FF, and RFamide related peptides.

In a particularly preferred embodiment, the artificially modified ligand is a modified peptide ligand of the neuropeptide Y1 receptor.

In other specific embodiments, the method further comprises releasing the drug molecule from the peptide ligand or protein ligand. Preferably, release is accomplished by means of cleaving the cleavable linker moiety.

In yet other specific embodiments, the G-protein coupled receptor is selected from the group consisting of the neuropeptide Y1, Y2, Y4 or Y5 receptor, gastrin releasing peptide receptor, neuromedin B receptor, orexin receptor 1 or 2, bradykinin receptor 1 or 2, melanocortin receptor 1, 2, 3 or 4, CXCR2 or CXCR4 receptor, endothelin receptor A or B, angiotensin II receptor, cholecystokinin receptor 1 or 2, and neuropeptide FF receptor 1 or 2.

In preferred embodiments, the method further comprises determining the internalization rate of the activated G-protein coupled receptor by using a fluorescently labeled G-protein coupled receptor and/or a fluorescently labeled peptide ligand or protein ligand, and particularly wherein the determination of the internalization rate is accomplished by means of fluorescence microscopy, fluorescence spectroscopy or an ELISA assay.

In other preferred embodiments, the method further comprises determining the internalization rate of the activated G-protein coupled receptor by using a radiolabeled G-protein coupled receptor and/or a radiolabeled peptide ligand or protein ligand, and particularly wherein the determination of the internalization rate is accomplished by means of scintillation counting of the radiolabel.

In other preferred embodiments, the activated G-protein coupled receptor is internalized to the endosomes and/or lysosomes of the one or more target cells. Specifically, the determination of the internalization rate of the activated G-protein coupled receptor further comprises the co-localization of the G-protein coupled receptor and/or the peptide ligand or protein ligand with lysosomal or late endosomal markers, and particularly wherein the lysosomal or late endosomal markers are selected from the group consisting of Rab7, Rab9, mannose-6-phosphate receptor, Lamp1, and Lamp2. Particularly preferably, the drug molecule is released from the peptide ligand or protein ligand intracellularly.

### BRIEF DESCRIPTION OF THE DRAWINGS

FIG. 1 shows the detection of recombinantly expressed NPY1 receptors by western blotting. 5 and 10 μg of recombinantly expressed protein were applied to SDS-PAGE and subsequent western blotting with to different anti-human NPY1 receptor antibodies (from USBiologicals and ABGENT, respectively) pAb, primary antibody.

FIG. 2 shows immunofluorescent staining of HEK293 cells stably expressing different NPY receptor subtypes (human Y1, Y2 or Y4 receptors) as well as SK-N-MC cells endogenously expressing NPY1 receptors. Cells were fixed and stained with anti-human NPY1 receptor primary antibody. Binding of the primary antibody to the NPY1 receptor was visualized by a DyLight-549 coupled secondary antibody (first panel). Cell nuclei were stained with HOECHST 33342 dye. Fluorescence from antibody-NPY1 receptor complex and cell nuclei was merged (last panel). Images were taken with an Axio Observer microscope and ApoTome image system (Zeiss, Jena, Germany). Scale bars: 20 μm.

FIG. 3 shows a cell surface ELISA to detect endogenous hY1R expression on the cell surface of SK-N-MC, T47D, MDA-MB231, MDA-MB468 and MCF-7 cells, respectively. HEK293 cells served as control. SK-N-MC cells had the highest hY1R surface expression, followed by T47D and MCF-7, which have similar hY1R levels. Expression of the hY1R could not be detected in MDA-MB231 and MDA-MB468 cells.

FIG. 4 shows the internalization of the human NPY1 and NPY2 receptor mediated by their native ligand NPY, the NPY1 receptor selective peptide [F⁷, P³⁴]-NPY and the NPY1 receptor selective drug conjugate CytoPep. HEK cells stably expressing the human NPY1 and NPY2 receptor (NPY1R and NPY2R, respectively) were treated with 1 μM peptide for 1 hour. Cell nuclei were stained with HOECHST33342. Live cell images were taken with an AxioObserver microscope with ApoTome imaging system (Zeiss, Jena, Germany).

FIG. 5 shows signal transduction of the human NPY1 and NPY2 receptor activated by the native ligand NPY and the peptide-drug conjugate CytoPep, respectively. Dose response curves for NPY and CytoPep were measured by IP₃ assay (FIG. 5A) and reporter gene assay (FIG. 5B).

FIG. 6 shows the endogenous expression of the NPY Y1 receptor (mRNA level) in various cell lines as determined by RT-qPCR using the GAPDH gene as reference. Data were analyzed by using the ΔΔCₜ methodology, and normalized to the receptor expression level of MDA-MB-468 cells.

FIG. 7 shows the inhibition of cell proliferation of (A) MDA-MB-468 breast cancer cells, and (B) SK-N-MC cells of the Ewing's sarcoma family. Cells were initially treated for 6 hours with different variants of the peptide-drug conjugate CytoPep. After cell proliferation in compound-free medium for 72 hours, cell viability was detected using a resazurin-based cell assay. The effects of the peptide-drug conjugates are expressed as IC₅₀ values.

### DETAILED DESCRIPTION OF THE INVENTION

The present invention is related to the unexpected finding that selecting a differentially expressed peptide- or protein-activated G-protein coupled receptor and determining its internalization rate does not only represent an accurate method for diagnosing a G-protein coupled receptor-related disease in one or more target cells but concomitantly also provides for an efficient means for transporting drug molecules (conjugated to the receptor ligand) to the site of therapeutic intervention. Such approach enables the use of drugs having a significantly reduced half-life, and thus results in a superior therapeutic window.

The present invention will be described in the following with respect to particular embodiments and with reference to certain drawings but the invention is to be understood as not limited thereto but only by the appended claims. The drawings described are only schematic and are to be considered non-limiting.

Where the term “comprising” is used in the present description and claims, it does not exclude other elements or steps. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Where an indefinite or definite article is used when referring to a singular noun e.g. “a”, “an” or “the”, this includes a plural of that noun unless specifically stated otherwise.

In case, numerical values are indicated in the context of the present invention the skilled person will understand that the technical effect of the feature in question is ensured within an interval of accuracy, which typically encompasses a deviation of the numerical value given of ±10%, and preferably of ±5%.

Furthermore, the terms first, second, third, (a), (b), (c), and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Further definitions of term will be given in the following in the context of which the terms are used. The following terms or definitions are provided solely to aid in the understanding of the invention. These definitions should not be construed to have a scope less than understood by a person of ordinary skill in the art.

The present invention relates to a method for diagnosing a G-protein coupled receptor-related disease in one or more target cells, comprising:

selecting a G-protein coupled receptor, the receptor being characterized in that it is:
(i) differentially expressed in the target cells as compared to healthy control cells, wherein the expression level in the target cells is at least 10 times the expression level in the control cells;
(ii) activated by a peptide ligand or a protein ligand; and
(iii) upon activation by binding of a ligand efficiently internalized into the one or more target cells together the peptide ligand or protein ligand,

wherein an internalization of at least 30% of the G-protein coupled receptor initially present in the cell membrane of the one or more target cells within less than 30 minutes after activation is indicative for the diagnosis of a G-protein coupled receptor-related disease.

In specific embodiments, the method is performed as an in vitro or ex vivo method.

The term “target cell”, as used herein, refers to any cell susceptible to be targeted by a peptide- or protein-activated G-protein-coupled receptor, that is, cells in which such receptors internalize. The term “one or more”, as used herein, is to be understood not only to include individual cells but also tissues, organs, and organisms.

In specific embodiments, the method is performed as an in vitro or ex vivo method.

The one or more target cells may be part of a sample derived from a subject, typically a mammal such as a mouse, rat, hamster, rabbit, cat, dog, pig, cow, horse or monkey, and preferably a human. Such samples may include body tissues (e.g., biopsies or resections) and body fluids, such as blood, sputum, and cerebrospinal fluid. The samples may contain a single cell, a cell population (i.e. two or more cells) or a cell extract derived from a body tissue, and may be used in unpurified form or subjected to any enrichment or purification step(s) prior to use. The skilled person is well aware of various such purification methods (see, e.g., Sambrook, J., and Russel, D. W. (2001), supra; Ausubel, F. M. et al. (2001) Current Protocols in Molecular Biology, Wiley & Sons, Hoboken, N.J., USA).

In preferred embodiments, the one or more target cells are disease cells, that is, cells that are dysfunctional as compared to healthy control cells. The diseases referred to herein are related to or mediated by G-protein-coupled receptors (also designated as seven-transmembrane helical receptors), which are well established in the art.

In specific examples, the one or more target cells are cells suspected to be tumor cells. The term “tumor” (also referred to as “cancer”), as used herein, generally denotes any type of malignant neoplasm, that is, any morphological and/or physiological alterations (based on genetic re-programming) of target cells exhibiting or having a predisposition to develop characteristics of cancer as compared to unaffected (healthy) control cells. Examples of such alterations may relate inter alia to cell size and shape (enlargement or reduction), cell proliferation (increase in cell number), cell differentiation (change in physiological state), apoptosis (programmed cell death) or cell survival. Exemplary tumor cells include inter alia those derived from breast cancer, colorectal cancer, prostate cancer, ovarian cancer (e.g., ovarian adenocarcinomas), leukemia, lymphomas, neuroblastoma, glioblastoma, melanoma, nephroblastoma, gastrointestinal stomal tumors, liver cancer, and lung cancer.

In other specific examples, the one or more target cells are suspected to be derived from an immune disease state. The term “immune disease”, as used herein, refers to any disorder of the immune system. Examples of such immune diseases include inter alia immunodeficiencies (i.e. congenital or acquired conditions in which the immune system's ability to fight infectious diseases is compromised or entirely absent, such as AIDS or SCID), hypersensitivity (such as allergies or asthma), and autoimmune diseases. The term “autoimmune disease”, as used herein, is to be understood to denote any disorder arising from an overactive immune response of the body against endogenic substances and tissues, wherein the body attacks its own cells.

Examples of autoimmune diseases include inter alia multiple sclerosis, Crohn's disease, lupus erythematosus, myasthenia gravis, rheumatoid arthritis, and polyarthritis.

In yet other specific examples, the one or more target cells are suspected to be derived from a cardiovascular disease state. The term “cardiovascular disease”, as used herein, refers to any disorder of the heart and the coronary blood vessels. Examples of cardiovascular diseases include inter alia coronary heart disease, angina pectoris, arteriosclerosis, cardiomyopathies, myocardial infarction, ischemia, and myocarditis.

In yet other specific examples, the one or more target cells are suspected to be derived from a neuronal disease state. The term “neuronal disease” (or “neurological disorder), as used herein, refers to any disorder of the nervous system including diseases of the central nervous system (CNS) (i.e. brain and spinal cord) and diseases of the peripheral nervous system. Examples of CNS diseases include inter alia Alzheimer's disease, Parkinson's disease, Huntington's disease, Locked-in syndrome, and Tourettes syndrome. Examples of diseases of the peripheral nervous system include, e.g., mononeuritis multiplex and polyneuropathy.

The term “G-protein coupled receptor”, as used herein, refers to those members of this receptor family that are activated by a proteinaceous ligand, that is, a peptide ligand or a protein ligand such as inter alia cytokines, peptide hormones and neuropeptides. In specific embodiments, the G-protein coupled receptor is selected from the group consisting of the neuropeptide Y1, Y2, Y4 or Y5 receptor, gastrin releasing peptide receptor, neuromedin B receptor, orexin receptor 1 or 2, bradykinin receptor 1 or 2, melanocortin receptor 1, 2, 3 or 4, CXCR2 or CXCR4 receptor, endothelin receptor A or B, angiotensin II receptor, cholecystokinin receptor 1 or 2, and neuropeptide FF receptor 1 or 2. The skilled person is well aware how as to select other such G-protein coupled receptors being activated by a proteinaceous ligand.

The method may be performed by analyzing a single G-protein coupled receptor (GPCR), a GPCR homodimer, a GPCR heterodimer or by concomitantly analyzing two or more different GPCRs (present as monomers and/or dimers).

According to the present invention, the G-protein coupled receptor is differentially expressed in the one or more target cells as compared to the one or more control cells. In particular, the expression level of the receptor is higher in the target cells as compared to the control cells. The expression level may be determined at mRNA level or at protein level. The skilled person is well aware of various methods for determining the expression level, such as quantitative RT-PCR or Western blot analysis (see also, e.g., Ausubel, F. M. et al. (2001) Current Protocols in Molecular Biology, Wiley & Sons, Hoboken, N.J., USA; Sambrook, J., and Russel, D. W. (2001), Molecular cloning: A laboratory manual (3rd Ed.) Cold Spring Harbor, N.Y., Cold Spring Harbor Laboratory Press). The expression level of the G-protein coupled receptor may be three times, five times or eight times higher in the one or more target cells than in the one or more control cells. Typically, the expression level of the G-protein coupled receptor is at least ten times higher in the one or more target cells than in the one or more control cells, that is, for example, ten times, twelve times, 15 times, 18 times, 20 times, 25 times, and so forth higher in the one or more target cells than in the one or more control cells.

In preferred embodiments, the peptide ligand or protein ligand employed in the present invention is conjugated to a drug molecule, and particularly wherein conjugation is accomplished by means of a cleavable linker moiety or a non-cleavable linker moiety.

Virtually any drug molecule may be used in connection with the present invention, for example, a cytotoxic or an anti-inflammatory molecule. Particular examples include inter alia tubulysins and derivatives thereof, natural and synthetic epothilones and derivatives thereof, auristatins, dolastatins, natural and synthetic vincristine and its analogues, natural and synthetic vinblastine and its analogues, amanitine and its analogues, maytansines and its analogues, taxanes, Nemorubicin, PNU-159682, pyrrolobenzodiazepins and dimers, duocarmycins and its analogues. The skilled person is well aware how as to select other drug molecules that can be employed in the present invention.

Typically, the drug molecule employed has a cellular activity of less than 500 nM or less than 400 nM, preferably of less than 300 nM or less than 200 nM, and particularly preferably of less than 100 nM or less than 50 nM (e.g., 100 nM, 90 nM, 80 nM, 70 nM, 60 nM, 50 nM, 40 nM, 30 nM, 20 nM or 10 nM). Typically, the drug molecule employed has a half-life of less than 24 hours or less than 12 hours, preferably of less than 8 hours or less than 6 hours, more preferably of less than 4 hours or less than 2 hours, and particularly preferably of less than 1 hour or less than 30 min (e.g., 60 min, 50 min, 40 min, 30 min, 20 min, 10 min).

Receptor ligand and drug molecule may be conjugated to each by a covalent or a non-covalent linkage. The term “covalent linkage” refers to an intra-molecular form of chemical bonding characterized by the sharing of one or more pairs of electrons between two components, producing a mutual attraction that holds the resultant molecule together. The term “non-covalent linkage” refers to a variety of interactions that are not covalent in nature, between molecules or parts of molecules that provide force to hold the molecules or parts of molecules together usually in a specific orientation or conformation. Such non-covalent interactions include inter alia ionic bonds, hydrophobic interactions, hydrogen bonds, Van-der-Waals forces, and dipole-dipole bonds.

In case of a covalent linkage, receptor ligand and drug molecule are typically conjugated via a linker molecule that serves to physically separate the peptide of the invention and the at least one other moiety and thus to ensure that neither entity is limited in their function due to the close vicinity to the other. Depending on the drug molecule employed, the linker may be, e.g., a peptide bond, an amino acid, a peptide of appropriate length, or a different molecule providing the desired features. In specific embodiments, the linker is a lysine or an arginine residue whose ε-amino groups are suitable to couple the peptides as defined herein to various other moieties. The linker moiety may be cleavable (e.g. enzymatically) or non-cleavable. The skilled person knows how to design appropriate linker molecules, in particular linker peptides based on his common knowledge. For example, peptide linkers can be chosen from the LIP (Loops in Proteins) database (Michalsky, E. et al. (2003) Prot. Eng. 56, 979-985). Such linker may be attached to the N- or the C-terminus or, if deemed suitable, also to a non-terminal amino acid residue of the peptide of the present invention.

In specific embodiments, the peptide ligand or protein ligand is a naturally occurring ligand of the G-protein coupled receptor. Preferably, the naturally occurring ligand is selected from the group consisting of cytokines, peptide hormones and neuropeptides, and particularly preferably selected from the group consisting of neuropeptide Y, peptide YY, pancreatic polypeptide, orexin A, orexin B, gastrin releasing peptide, bombensin, litorin, neuromedin B, neuromedin C, endothelin-1, endothelin-3, SDF-1, GROα, IL-8, melanocortin peptides, angiotensin II, bradykinin, cholestocytokinin, neuropeptide FF, and RFamide related peptides. The skilled person is well aware how as to select other naturally occurring G-protein coupled receptors ligands that can be employed in the present invention.

In other specific embodiments, the peptide ligand or protein ligand is an artificially modified ligand. Preferably, the artificially modified ligand is based on a naturally occurring ligand being selected from the group consisting of cytokines, peptide hormones and neuropeptides, and particularly preferably selected from the group consisting of neuropeptide Y, peptide YY, pancreatic polypeptide, orexin A, orexin B, gastrin releasing peptide, bombensin, litorin, neuromedin B, neuromedin C, endothelin-1, endothelin-3, SDF-1, GROα, IL-8, melanocortin peptides, angiotensin II, bradykinin, cholestocytokinin, neuropeptide FF, and RFamide related peptides. In a particularly preferred embodiment, the artificially modified ligand is a modified peptide ligand of the neuropeptide Y1 receptor. The skilled person is well aware how as to introduce one or more artificial modifications into a ligand molecule, for example by means of recombinant DNA technology and expression of the modified molecules or by chemical modification. Such artificial modifications may include the addition, deletion or substitution of one or more amino acid residues and/or the post-translational modifications of amino acid residues by acetylation, palmitoylation, HESylation, PEGylation, PARylation, or the like (see also, e.g., Ausubel, F. M. et al. (2001) Current Protocols in Molecular Biology, Wiley & Sons, Hoboken, N.J., USA; Sambrook, J., and Russel, D. W. (2001), Molecular cloning: A laboratory manual (3rd Ed.) Cold Spring Harbor, N.Y., Cold Spring Harbor Laboratory Press).

In other specific embodiments, the method further comprises releasing the drug molecule from the peptide ligand or protein ligand. Preferably, release is accomplished by means of cleaving the cleavable linker moiety such as by using enzymes recognizing specific cleavage site located in the linker moiety.

Upon activation by binding of then peptide ligand or the protein ligand, the G-protein coupled receptor employed is efficiently internalized into the one or more target cells together the peptide ligand or protein ligand, wherein an internalization of at least 30% of the G-protein coupled receptor initially present in the cell membrane of the one or more target cells within less than 30 minutes after activation is indicative for the diagnosis of a G-protein coupled receptor-related disease.

The term “internalization”, as used herein, refers to the ability of G-protein coupled receptor (in complex with its ligand bound thereto) to pass cellular membranes (including inter alia the outer cell membrane (also commonly referred to as “plasma membrane”), endosomal membranes, and membranes of the endoplasmatic reticulum) and/or to direct the passage of a given ligand-drug conjugate to these cellular membranes. In the context of the present invention, any possible mechanism of internalization is envisaged including both energy-dependent (i.e. active) transport mechanisms (e.g., endocytosis) and energy-independent (i.e. passive) transport mechanism (e.g., diffusion). As used herein, the term “internalization” is to be understood as involving the localization of at least a part of the G-protein coupled receptor being localized in the cellular membrane into the cytoplasma.

Receptor mediated (or related) endocytosis of macromolecules includes the action of clathrin-coated pits as segments of the cell membrane that is specialized for receptor-related endocytosis. In case of a ligand mediated receptor activation the plasma membrane is shaped into clathrin coated vesicles that immediately uncoat and fuses with endosomes. The endosome functions as a switching area that directs membrane and content molecules to specific locations within the cell. The role of receptor-mediated endocytosis is also well recognized in the down-regulation of transmembrane signal transduction. The activated receptor may become internalized into early endosomes and is transported to late endosomes and further to lysosomes for degradation (reviewed, e.g., in Rappoport (2008) Biochem. J. 412, 415-423).

Other mechanisms of transporting molecules into cells include macropinocytosis, non-specific adsorptive pinocytosis, and phagocytosis.

The term “internalization efficacy”, as used herein, is to be understood in a broad sense. The term does not only refer to the extent to which G-protein coupled receptor (along with its ligand and optionally a drug molecule conjugated thereto) passes through the plasma membrane of cells (i.e. the internalization behavior per se) but also to the efficiency by which the G-protein coupled receptor/ligand-complex directs the passage of a given drug molecule through the cell plasma membrane. Numerous methods of determining the internalization behavior are established in the art, for example, by attaching a detectable label (e.g. a fluorescent dye) to the G-protein coupled receptor and/or to the peptide or protein ligand or by fusing the peptide or protein ligand with a reporter molecule, thus enabling detection once cellular uptake occurred.

Detectable labels that may be used herein include any compound, which directly or indirectly generates a detectable compound or signal in a chemical, physical or enzymatic reaction. Labeling and subsequent detection can be achieved by methods well known in the art (see, for example, Sambrook, J., and Russel, D. W. (2001), supra; Ausubel, F. M. et al. (2001), supra; and Lottspeich, F., and Zorbas H. (1998) Bioanalytik, Spektrum Akademischer Verlag, Heidelberg/Berlin, Germany). The labels can be selected inter alia from fluorescent labels, enzyme labels, chromogenic labels, luminescent labels, radioactive labels, haptens, biotin, metal complexes, metals, and colloidal gold, with fluorescent labels being preferred. All these types of labels are well established in the art and can be commercially obtained from various suppliers. An example of a physical reaction that is mediated by such labels is the emission of fluorescence or phosphorescence upon irradiation. Alkaline phosphatase, peroxidase, β-galactosidase, and β-lactamase are examples of enzyme labels, which catalyze the formation of chromogenic reaction products, and which may be used in the invention. Label detection may occur inter alia by means of FACS analysis fluorescence spectroscopy or via specific antibodies such as via an ELISA assay (see, e.g., Ausubel, F. M. et al. (2001) Current Protocols in Molecular Biology, Wiley & Sons, Hoboken, N.J., USA). The skilled person is also well aware how to select the respective concentration ranges of the peptide or protein ligand and, if applicable, of the drug molecule to be employed in such methods, which may depend on the nature of the peptide or protein ligand, the size of the drug molecule, the cell type used, and the like.

An internalization efficiency of at least 30% (e.g. at least 35%, at least 40%, at least 45%, at least 50%, at least 55%, at least 60%, at least 65%, at least 70%, at least 75%, at least 80%, at least 85%, at least 90%, at least 95% or 100%) of the G-protein coupled receptor initially present in the cell membrane of the one or more target cells being re-located within less than 30 minutes after activation is indicative for the diagnosis of a G-protein coupled receptor-related disease. In other embodiments, the time period for accomplishing receptor re-location is less than 6 hours, less than 5 hours, less than 4 hours, less than 3 hours, less than 2 hours, less than 1 hour, less than 45 min or less than 15 min.

In preferred embodiments, the method further comprises determining the internalization rate of the activated G-protein coupled receptor by using a fluorescently labeled G-protein coupled receptor and/or a fluorescently labeled peptide ligand or protein ligand, and particularly wherein the determination of the internalization rate is accomplished by means of fluorescence microscopy, fluorescence spectroscopy or an ELISA assay.

In other preferred embodiments, the method further comprises determining the internalization rate of the activated G-protein coupled receptor by using a radiolabeled G-protein coupled receptor and/or a radiolabeled peptide ligand or protein ligand, and particularly wherein the determination of the internalization rate is accomplished by means of scintillation counting of the radiolabel.

In other preferred embodiments, the activated G-protein coupled receptor is internalized to the endosomes and/or lysosomes of the one or more target cells. Specifically, the determination of the internalization rate of the activated G-protein coupled receptor further comprises the co-localization of the G-protein coupled receptor and/or the peptide ligand or protein ligand with lysosomal or late endosomal markers, and particularly wherein the lysosomal or late endosomal markers are selected from the group consisting of Rab7, Rab9, mannose-6-phosphate receptor, Lamp1, and Lamp2. Particularly preferably, the drug molecule is released from the peptide ligand or protein ligand intracellularly.

The invention is further described by the figures and the following examples, which are solely for the purpose of illustrating specific embodiments of this invention, and are not to be construed as limiting the claimed subject matter in any way.

### EXAMPLES

### Example 1: Selection of a Disease Related G-Protein Coupled Receptor for Efficient Drug Transport

It has been shown that the NPY-1 receptor is overexpressed in certain types of cancer such as breast cancer, especially metastatic breast cancer, but also in Ewing's sarcoma, renal cell carcinomas, gastrointestinal stromal tumors, nephroblastomas, neuroblastic tumors, paragangliomas, pheochromocytomas, adrenal cortical tumors, ovarian sex cord-stromal tumors, and ovarian adeno carcinomas (Kömer and Reubi (2007) Peptides 28, 419-425). The NPY1 receptor, upon activation by its natural ligand, the NPY peptide, internalizes. Zwanziger and coworkers (Zwanziger et al. (2008) Bioconjugate Chem. 19, 1430-1438), used a chelator bound to modified NPY peptides for diagnostic purposes. The half-life of some conjugates was found to be much longer (>24 h) as compared to the native peptide (about 4 minutes) in different tissues. For example, the modified NPY included a change in position 7 to phenylalanine and in position 34 to proline. This modified NPY molecule showed selectivity for the NPY1 receptor over the competing NPY2, NPY4, or NPY5 receptors. On the other hand, however, the conjugates exhibited a very low uptake by tumor cells.

NPY1 ligand-toxin conjugates, using daunorubicin and doxorubicin as cytotoxic drugs, were shown to be able to bind to the receptor with affinities ranging from 25 to 51 nM, but exhibited no activity in vivo. (Langer et al. (2001) J. Med. Chem. 44, 1341-1348).

### Example 2: Cloning of Receptor cDNA Sequences

Detailed analysis of ligand-receptor or drug-receptor interactions require the molecular cloning of the respective target receptor. All techniques described herein in connection with the cloning of the NPY1 receptor are based on establish standard protocols and thus transferable to the cloning of virtually any other receptor.

The cDNA of the human NPY1 receptor was amplified with specific forward and reverse primers covering the complete coding sequence of the receptor by polymerase chain reaction (PCR) using Phusion polymerase. The PCR product was purified by agarose gel electrophoresis and commercially available purification kits (e.g. Wizard SV Gel & PCR Clean-up Kit by Promega, Mannheim, Germany). The PCR product of the cDNA of the human NP Y1 receptor was then cleaved by restriction enzymes BamHI and MluI at the cognate recognition sites provided by the PCR primers. Cleaved PCR products were purified using commercially available purification kits as described above. The PCR product of the human NP Y1 receptor was sub-cloned into the eukaryotic expression vector pVitro2-mcs (Invivogen), which was modified to encompass an enhanced yellow fluorescent protein (EYFP) in its multiple cloning site (EYFP-pVitro). The EYFP-pVitro vector includes the same restriction sites on the 5′ end of the EYFP cDNA as present in the PCR product of the NPY receptor. The cleaved PCR product of the NPY Y1 receptor was then ligated into the BamHI/MluI-cleaved EYFP-pVitro by T4 ligase to obtain an eukaryotic expression plasmid containing the human NPY receptor being C-terminally fused (in frame) to EYFP (hY1R-EYFP-pVitro). This fusion was constructed such that the NPY receptor sequence and the EYFP sequence were separated by a short spacer sequence (typically 6 to 10 nucleotide triplets) to ensure correct folding of the resulting proteins expressed in eukaryotic cells. In order to determine the cell surface expression of NPY receptor-EYFP fusion proteins, an immunogenic hemagglutinin (HA) tag was inserted N-terminally to the NPY receptor. Integrity of all plasmids was checked by DNA sequencing.

### Example 3: Stable Transfection of Receptor Plasmids in Eukaryotic Cells for In Vitro Cell-Based Screenings

For stable expression of NPY receptor-EYFP constructs in eukaryotic cells, the receptor plasmid needs to be stably integrated into the genome of these cells. The stable transfection of HEK293 cells with NPY receptors followed mainly the protocol described in Böhme et al. (Böhme et al. (2008) Cell. Signal. 20, 1740-1749).

For human NPY1 receptor, the hY1R-EYFP-pVitro plasmid was linearized by using restriction enzyme NheI and purified with commercially available kits (e.g. Wizard SV Gel & PCR Clean-up Kit by Promega, Mannheim, Germany). HEK293 cells were transfected with 4 μg linearized plasmid and 15 μl Metafectene® Pro transfection reagent (Biotex Laboratories GmbH, Martinsried, Germany) according to manufacturer's instructions. Cells were then allowed to grow for two days. Cells were then transferred into a 10 cm petri dish and, subsequently, selection of clones started with medium containing 100 μg/ml hygromycin B (Merck KgaA, Darmstadt, Germany). Medium was changed every two to three days. After 10 to 12 days, clones were selected according to their fluorescence and transferred into 96 well-plates. In the following days, clones were expanded still under selection pressure of medium containing 100 μg/ml hygromycin B and then transferred into the next higher well format (24 well, 6 well and 25 cm² culture flask). Stable transfection was examined by fluorescence microscopy and detection of Y receptor expression by PCR. All techniques described herein for the stable transfection of cells with NPY1 receptor plasmid are generally transferable for the usage of each other receptor gene in the pVitro plasmid.

### Example 4: Receptor Detection by Western Blotting

To detect expression of the human NPY1 receptor, western blotting was applied. Recombinantly expressed and purified human NPY1 receptor was loaded with 5 or 10 μg protein on a SDS-PAGE gel (4% stacking gel and 10% running gel) and was separated by 20 mA for 45-60 minutes. Subsequently, proteins were blotted on a PVDF membrane (GE Healthcare, Munich, Germany) over night by constant current of 5 V. The blotting membrane was blocked in 5% BSA/TBS-T for 1 hour at room temperature. Incubation with the primary antibody (mouse anti-human NPY1 receptor antibody from USBiological, Salem, USA or ABGENT Europe, Oxfordshire, UK) was performed overnight at 4° C. with an antibody concentration of 1:500 in 5% BSA/TBS-T. Subsequently, the blot membrane was washed trice with PBS and was incubated with secondary antibody (donkey anti-mouse IgG-HRP, SantaCruz, Heidelberg, Germany) at 1:5000 in 0.2% BSA/TBS-T for 2 hours. Blotting membrane was washed in triplicate with PBS and incubated with ECL detection solution (Thermo Scientific, Bonn, Germany). Pictures were taken with the G:Box Gel Documentation System (Syngene Europe, Cambridge, UK).

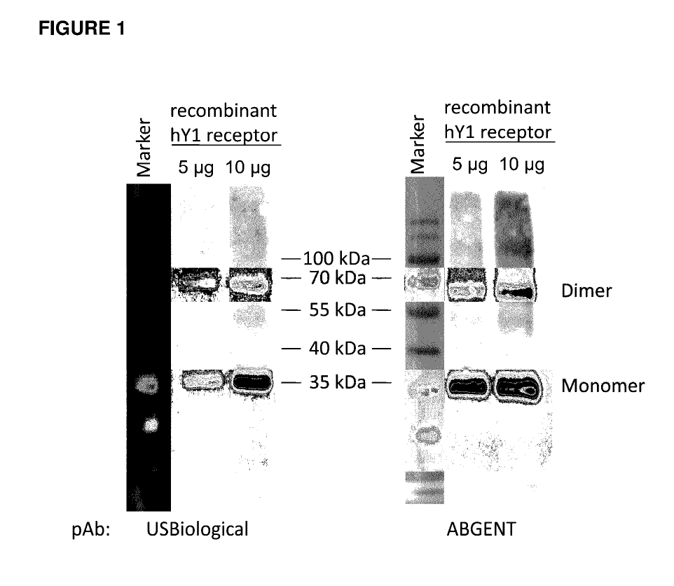

Western blotting of recombinantly expressed and purified human NPY1 receptor showed, that these receptors are detectable with specific anti-NPY1 receptor antibodies. Both the monomeric and the dimeric receptor form could be detected (FIG. 1).

FIG. 1 shows the detection of recombinantly expressed NPY1 receptors by western blotting. 5 and 10 μg of recombinantly expressed protein were applied to SDS-PAGE and subsequent western blotting with to different anti-human NPY1 receptor antibodies (from USBiologicals and ABGENT, respectively) pAb, primary antibody.

### Example 5: Receptor Detection by Immunofluorescence

To detect subcellular localization of the human NPY1 receptor, immunofluorescence was applied. HEK293 cells stably expressing the human NPY1 receptor fused C-terminally to EYFP (HEK-hY1R-EYFP) were seeded with 250000 cells/well into sterile μ-slide 8 well ibidi-plates (Ibidi GmbH, Martinsried, Germany). HEK293 cells stably expressing the human NPY2 or NPY4 receptor fused C-terminally to EYFP (HEK-hY2R-EYFP and HEK-hY4R-EYFP, respectively) served as controls and were treated the same way as HEK-hY1R-EYFP. SK-N-MC cells were used to detect the expression of the endogenous NPY1 receptor. Cells were fixed in 2% PFA for 20 minutes at room temperature and were then washed trice with PBS. Cells were blocked in 10% BSA/PBS for 1 hour at room temperature. Subsequently, cells were incubated with primary mouse anti-human NPY1 receptor antibody (USBiological, Salem, USA) in a concentration of 1:25 in 5% BSA/PBS for 2 hours at 37° C. Cells were then washed trice with PBS and incubated with secondary rabbit anti-mouse IgG coupled to DyLight 549 (Rockland, Gilbertsville, USA) with a concentration of 1:1000 in 5% BSA/PBS. Cells were washed trice with PBS and images were taken with an Axio Observer microscope and ApoTome imaging system (Zeiss, Jena, Germany). Microscopy images were analyzed with the Zeiss Axio Viosion software Release 3.0.

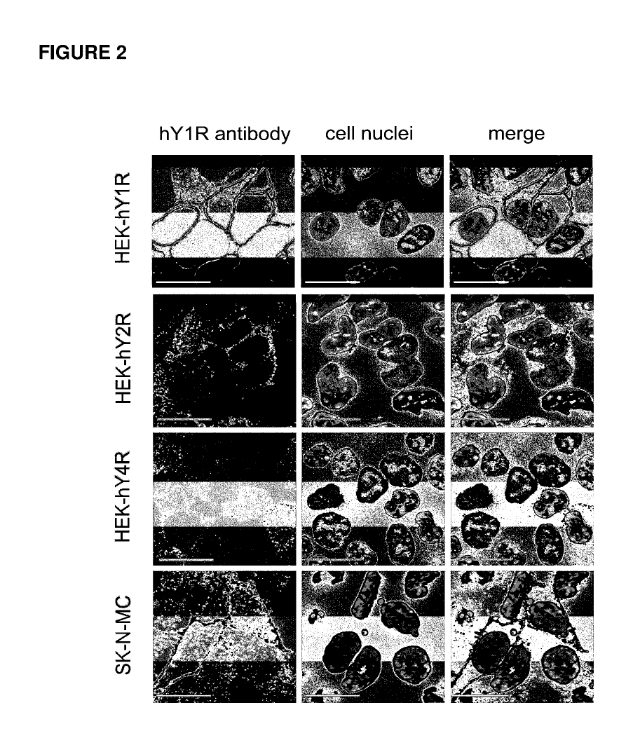

In order to detect and localize the expression of the human NPY1 receptor in the cell membrane, we subjected HEK293 cells that stably express the human NPY1, NPY2 and NPY4 receptor, respectively, and SK-N-MC that express the NPY1 receptor endogenously to immunofluorescent staining. FIG. 2 shows (1) that the human NPY Y1 receptor is localized in the cell membrane of cells expressing that receptor, (2) that the primary anti-human NPY1 receptor antibody specifically recognizes the human NPY1 receptor, but not other NPY receptor subtypes, and (3) that it is also possible to detect the endogenously, and therefore weaker, expressed receptor in SK-N-MC cells. Consequently, this satisfies the criteria necessary for a diagnostic tool.

FIG. 2 shows immunofluorescent staining of HEK293 cells stably expressing different NPY Y receptors (human Y1, Y2 or Y4 receptors) as well as SK-N-MC cells endogenously expressing NPY Y1 receptors. Cells were fixed and stained with anti-human NPY Y1 receptor primary antibody. Binding of the primary antibody to the NPY Y1 receptor was visualized by a DyLight-549 coupled secondary antibody (first panel). Cell nuclei were stained with HOECHST 33342 dye. Fluorescence from antibody-NPY Y1 receptor complex and cell nuclei was merged (last panel). Images were taken with an Axio Observer microscope and ApoTome image system (Zeiss, Jena, Germany). Scale bars: 20 μm.

### Example 6: Cell Surface Localization by ELISA

To ensure receptor localization in the plasma membrane and to compare receptor densities between different cell types, a cell surface ELISA was used to detect NPY1 receptor expression. Endogenous NPY receptor expression was investigated by seeding HEK293, SK-N-MC, T47D, MDA-MB231, MDA-MB468 and MCF-7 cells into 96 well-plates with 100000 cells/well. Cells were incubated for at least 24 hours at 37° C./5% CO₂ in humidified atmosphere. Subsequently, cells were fixed with 4% PFA for 20 minutes at room temperature, washed trice with PBS and blocked in cell culture medium supplemented with 15% FCS for 1 hour at 37° C. Subsequently, cells were washed trice with PBS and incubated with primary mouse anti-human NPY1 receptor antibody (USBiological, Salem, USA) in a concentration of 1:50 in cell culture medium supplemented with 15% FCS for 2 hours at 37° C. Cells were then washed trice with PBS and incubated with rabbit anti-mouse IgG-HRP (SantaCruz Biotechnologies, Heidelberg, Germany) in a concentration of 1:1000 cell culture medium supplemented with 15% FCS for 1 hour at 37° C. HRP reaction was initialized by addition of 100 μl TMB substrate for 1-5 minutes. Reaction was stopped with 100 μl of 250 mM HCl. Absorbance was measured at 450 nm using the microplate reader Synergy2 (BioTek, Bad Friedrichshall, Germany).

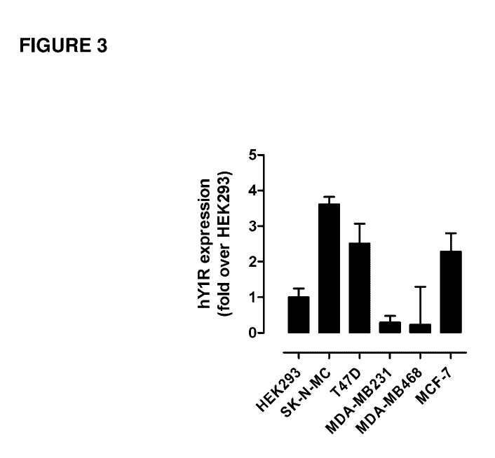

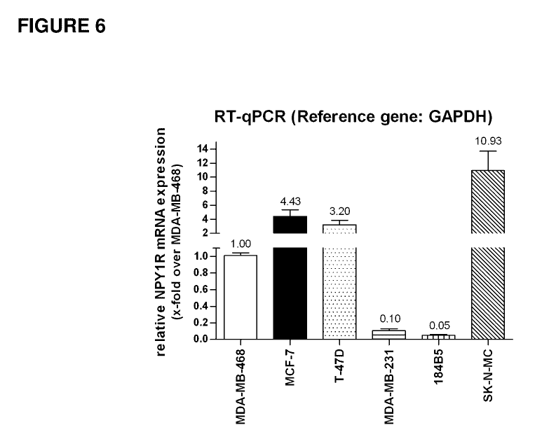

Cell surface ELISA to detect endogenous hY1R expression on the cell surface was done with SK-N-MC, T47D, MDA-MB231, MDA-MB468 and MCF-7 cells. HEK293 cells served as control. FIG. 3 shows that SK-N-MC cells have the highest hY1R surface expression, followed by T47D and MCF-7, which have similar hY1R levels on the cell surface. Expression of the hY1R could not be detected for MDA-MB231 and MDA-MB468 cells. This might correspond to the detection limit of the assay system. These data are well in accordance with the hY1R mRNA expression studies shown in FIG. 6.

### Example 7: Receptor Internalization Studies

HEK293 cells stably transfected with the human NPY1 receptor C-terminally fused to EYFP (HEK293-hY1R-EYFP) and the human NPY2 receptor C-terminally fused to EYFP and an HA tag (HEK293-HA-hY2R-EYFP) were seeded into sterile μ-slide 8 well-plates (ibidi GmbH, Martinsried, Germany) and incubated until 80% confluency was reached. Cells were incubated for 30 minutes in OptiMEM prior to ligand stimulation. Cell nuclei were stained with HOECHST 33342 nuclear dye. Cells were stimulated for 60 minutes with 1 μM NPY or the peptide-drug conjugate “CytoPep” in OptiMEM at 37° C. Live cell images were obtained with an Axio Observer microscope and ApoTome imaging system (Zeiss, Jena, Germany). Microscopy images were analyzed with the Zeiss Axio Vision software Release 3.0.

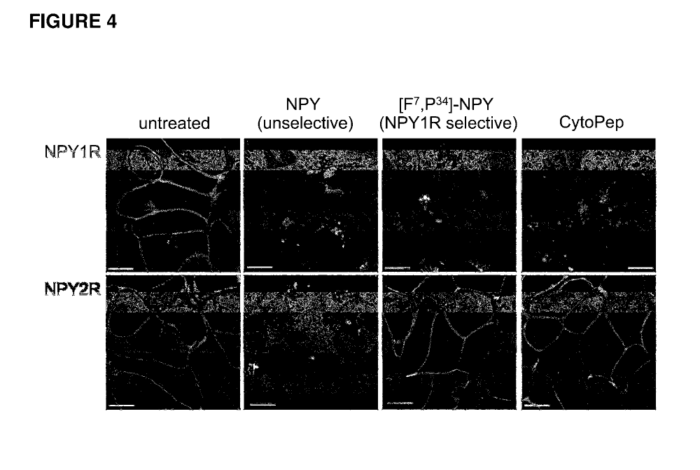

The aim of peptide-drug conjugates is to deliver the cytotoxic compound inside the cell via a specific receptor-mediated internalization process. As shown in FIG. 4, internalization studies of HEK293 cells stably expressing the human NPY1 or NPY2 receptor by fluorescence microscopy revealed that the NPY1 receptor selective peptide-drug conjugate CytoPep and the selective ligand [F⁷, P³⁴]-NPY induced only internalization of the human NPY1 receptor but not of the NPY2 receptor in contrast to the unselective ligand NPY.

FIG. 4 shows the internalization of the human NPY1 and NPY2 receptor mediated by their native ligand NPY, the NPY1 receptor selective peptide [F⁷, P³⁴]-NPY and the NPY1 receptor selective drug conjugate CytoPep. HEK cells stably expressing the human NPY1 and NPY2 receptor (NPY1R and NPY2R, respectively) were treated with 1 μM peptide for 1 hour. Cell nuclei were stained with HOECHST33342. Live cell images were taken with an AxioObserver microscope with ApoTome imaging system (Zeiss, Jena, Germany).

### Example 8: Functional Receptor Activation (Signal Transduction)

To evaluate the ability of the NPY-derived peptide-drug conjugates to functionally activate hY1R with high affinity, and to ensure proper hY1R selectivity compared to even closely related receptors, two different types of cell-based assays were performed. A functional IP₃ second messenger assay as well as a functional reporter gene assay (using cAMP response element—CRE).

For IP₃ second messenger assays, Cos-7 cells, stably transfected with the cDNA encoding the human Y1 receptor C-terminally fused to EYFP and the human NPY2 receptor C-terminally fused to EYFP as well as the chimeric G protein were seeded into 24 well-plates. 24 hours after seeding, cells were incubated for 16 hours with ³H-myo-inositol solution (300 μl DMEM/0.6 μl ³H-myo-inositol per well). Subsequently, cell culture medium was removed and cells were washed with 500 μl DMEM containing 10 mM LiCl. After 1 hour stimulation with different peptide concentrations (10⁻⁵ to 10⁻¹² M) in DMEM containing 10 mM LiCl was performed. ³H-inositol phosphates were accumulated. After stimulation, the samples were hydrolyzed with 150 μl 0.1N NaOH for 5 minutes. Neutralization was carried out by addition of 50 μl 0.2 M formic acid. The samples were subsequently diluted in IP dilution buffer and the cell pellet was removed with a pipette. ³H-inositol phosphates were isolated by anion exchange chromatography.

CRE reporter gene assays were performed by transiently co-transfecting CHO cells with cDNA encoding the human NPY1 receptor and NPY2 receptor, respectively, C-terminally fused to EYFP and the CRE reporter vector pGL4.29 (Promega GmbH, Mannheim, Germany). For this purpose, 2.5·10⁶ CHO cells were seeded per 25 cm² cell culture flask and allowed to adhere overnight. Subsequently, co-transfection of the cells was done using 10 μg hYxR vector, 2 μg pGL4.29 reporter vector and 25 μl of Metafectene® Pro transfection reagent (Biontex Laboratories GmbH, Martinsried, Germany) per culture flask. After 3 hours transfection in PBS under standard growth conditions, the transfection solution was discarded; transfected cells were detached and seeded in white/clear bottom 96-well plates at 50000 cells/well. In order to allow receptor and reporter gene expression, cells were cultured for 48 hours under standard growth conditions. Then, cells were co-stimulated with 10⁻⁶ M forskolin (adenylyl cyclase activator for cAMP elevation) and 10⁻¹¹-10⁻⁵M of the peptides/peptide-drug conjugates under investigation (reduction of cAMP levels by Gαi-mediated signal transduction of activated hYx receptors). After 6 hours stimulation at 37° C., incubation media were removed and 60 μl/96-well of Promega's ONE-Glo™ reagent (1:1 in DMEM, v/v) were added. After 10 min incubation at room temperature the reporter gene generated luminescence signal was measured by using a Synergy 2 multiwell plate reader (BioTek, Bad Friedrichshall, Germany).

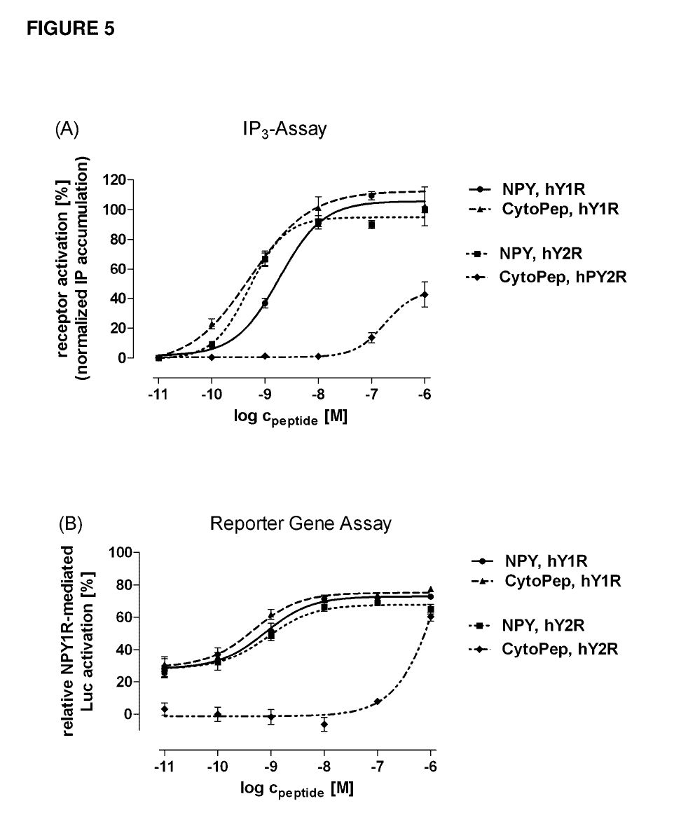

Functional assays to measure the activation of target receptors are necessary to (1) determine whether a drug conjugate is able to functionally address its target receptor and to (2) analyse the specificity of drug conjugates. FIG. 5 shows that the peptide-drug conjugate CytoPep specifically addresses the human NPY1 receptor with nanomolar potency but not the NPY2 receptor.

FIG. 5 shows signal transduction of the human NPY1 and NPY2 receptor activated by the native ligand NPY and the peptide-drug conjugate CytoPep, respectively. Dose response curves for NPY and CytoPep were measured by IP₃ assay (FIG. 5A) and reporter gene assay (FIG. 5B).

### Example 9: Receptor Expression Analysis Using RT-qPCR

Endogenous and ectopical receptor expression by several cell lines was analyzed by PCR techniques. Samples for expression analysis were prepared by RNA extraction using the Bio&Sell (Feucht, Germany) RNA Mini Kit and Qiagen's (Hilden, Germany) RNeasy Mini Kit, followed by a DNase I treatment and cDNA synthesis using RevertAid Premium Reverse Transcriptase (Fermentas, St. Leon-Rot, Germany). All methods were done according to the manufacturer's guidelines. Finally, receptor expression was analyzed by using appropriate primers and conventional PCR as well as quantitative real-time PCR (RT-qPCR) using a Bio-Rad (München, Germany) CFX96™ real-time PCR detection system. For qPCR, Bio-Rads SsoFast EvaGreen Supermix was used according to the manufacturer's guidelines.

Receptor expression analysis by RT-qPCR serves as control for expression levels of any receptor target, equally in transiently or stably transfected cells or cells endogenously expressing the receptor of interest, as shown in FIG. 6.

FIG. 6 shows the endogenous expression of the NPY Y1 receptor (mRNA level) in various cell lines as determined by RT-qPCR using the GAPDH gene as reference. Data were analyzed by using the ΔΔCₜ methodology, and normalized to the receptor expression level of MDA-MB-468 cells.

### Example 10: Cell Proliferation Assays

To evaluate the anti-proliferative and cytotoxic effect, respectively, of the peptide-drug conjugates, a fluorometric resazurin-based cell viability assay was used. Human cancer and non-cancer cell lines (primarily breast cancer) were seeded with low densities into 96-well plates (1500-20000 cells per well), and were allowed to adhere for 24 h. Subsequently, the compounds, dissolved to appropriate concentrations in medium, were added to the cells and incubated for 4-72 h. In case the compound treatment was shorter than 72 h, the incubation solution was discarded; cells were rinsed once with cell culture medium and were allowed to proliferate in compound-free medium until 72 h were reached. Subsequently, medium was replaced by 50 μM resazurin in medium, and the cells were incubated for 2 h. Finally, the conversion of resazurin to resorufin by viable, metabolically active cells was measured using a Synergy 2 multiwell plate reader (BioTek, Bad Friedrichshall, Germany) with 540 nm excitation and 590 nm emission filter setting.

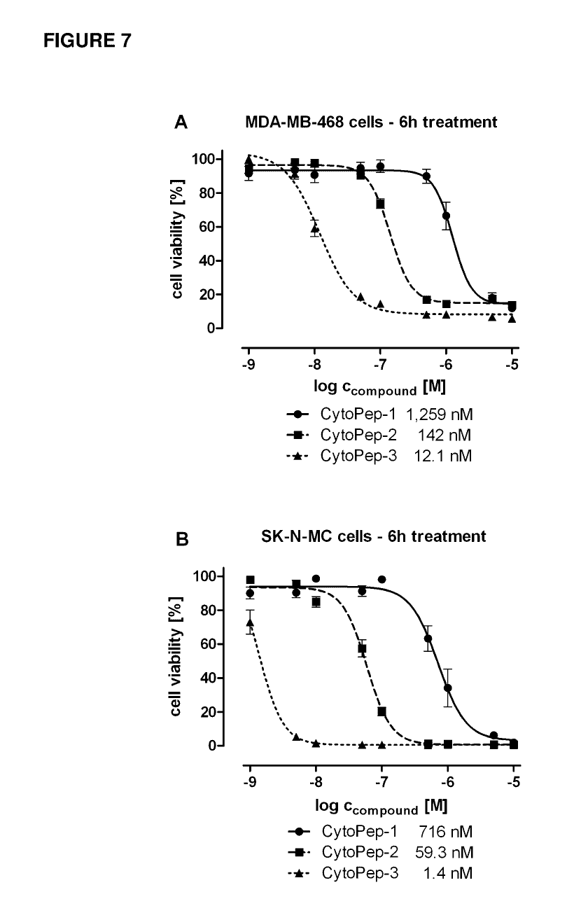

A fluorometric cell proliferation assay has been used to evaluate the cytostatic and cytotoxic, respectively, in vitro efficacy of the compounds under investigation, e.g. peptide-drug conjugates as the NPY Y1 receptor-selective CytoPep variants, as shown in FIG. 7. Using an appropriate cell line collection, preliminary decisions concerning, for instance, most sensitive cancer subtypes can be made. Combining cell proliferation data and data from receptor expression analysis, correlations of the receptor-targeting dependent biological effects and the receptor levels might be possible. Ideally, the determination of a distinct receptor expression threshold level, above that the treatment is promising, is possible.

FIG. 7 shows the inhibition of cell proliferation of (A) MDA-MB-468 breast cancer cells, and (B) SK-N-MC cells of the Ewing's sarcoma family. Cells were initially treated for 6 hours with different variants of the peptide-drug conjugate CytoPep. After cell proliferation in compound-free medium for 72 hours, cell viability was detected using a resazurin-based cell assay. The effects of the peptide-drug conjugates are expressed as IC₅₀ values.

### Example 11: Peptide-Drug-Conjugate CytoPep-3

Peptide H-Tyr-Pro-Ser-Lys(palmitoyl-Cys-βAla)-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Arg-Pro-Arg-Tyr-NH₂ was synthesized by standard solid phase synthesis methods according to the Fmoc/tBu strategy using an automated multiple solid-phase peptide synthesizer Syro II (MultiSynTech GmbH, Bochum, Germany). To gain C-terminal peptide amides, a Rink amide resin with a loading capacity of 0.63 mmol/g was used. The peptide is cleaved from the resin, by precipitation in ice cold diethyl ether and centrifugation at 4,400 g. The peptide was dried by using a SpeedVac, and finally lyophilized from 1-2 mL H₂O/tBuOH (1:3 v/v).

Coupling with the respective cytolysin derivative was performed via a disulfide linkage to the cysteine of K4(palmitoyl-Cys-βAla)-[F7,P34]-NPY, the purified peptide was dissolved in 0.1 mM phosphate buffer according to Sorensen (pH 6.0) and degased using argon. The coupling reaction was performed under equimolar conditions at room temperature. After 60 min the reaction was complete and product identity was confirmed by MALDI-TOF mass spectrometry. The product was purified immediately by preparative RP-HPLC.

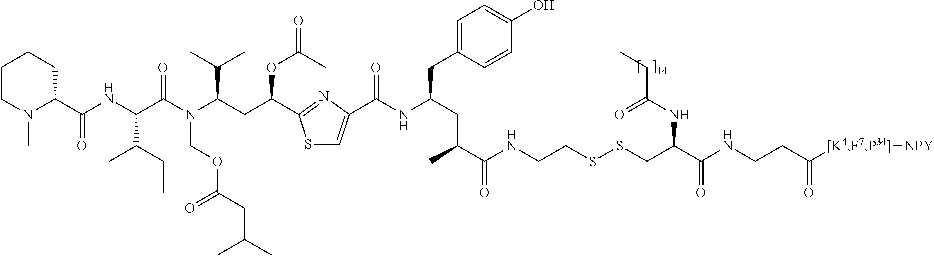

Calculated average molecular mass: 5569.52

Molecular formula: C262H397N61O67S3

MS-ESI: 929.1 [M+6H]⁶⁺

Data Analysis: For data analysis GraphPad Prism 5.03 and LibreOffice Calc were used.

The present invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modifications and variations of the inventions embodied therein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. Method for diagnosing a G-protein coupled receptor-related disease in one or more target cells, comprising:
selecting a G-protein coupled receptor, the receptor being characterized in that it is:
(i) differentially expressed in the target cells as compared to healthy control cells, wherein the expression level in the target cells is at least 10 times the expression level in the control cells;
(ii) activated by a peptide ligand or a protein ligand; and
(iii) upon activation by binding of a ligand efficiently internalized into the one or more target cells together the peptide ligand or protein ligand,
wherein an internalization of at least 30% of the G-protein coupled receptor initially present in the cell membrane of the one or more target cells within less than 30 minutes after activation is indicative for the diagnosis of a G-protein coupled receptor-related disease.

2. The method of claim 1, wherein the peptide ligand or protein ligand is conjugated to a drug molecule, and particularly wherein conjugation is accomplished by means of a cleavable linker moiety or a non-cleavable linker moiety.

3. The method of claim 1, wherein the peptide ligand or protein ligand is a naturally occurring ligand of the G-protein coupled receptor.

4. The method of claim 3, wherein the naturally occurring ligand is selected from the group consisting of cytokines, peptide hormones and neuropeptides, and particularly selected from the group consisting of neuropeptide Y, peptide YY, pancreatic polypeptide, orexin A, orexin B, gastrin releasing peptide, bombensin, litorin, neuromedin B, neuromedin C, endothelin-1, endothelin-3, SDF-1, GROα, IL-8, melanocortin peptides, angiotensin II, bradykinin, cholestocytokinin, neuropeptide FF, and RFamide related peptides.

5. The method of claim 1, wherein the peptide ligand or protein ligand is an artificially modified ligand.

6. The method of claim 5, wherein the artificially modified ligand is based on a naturally occurring ligand being selected from the group consisting of cytokines, peptide hormones and neuropeptides, and particularly selected from the group consisting of neuropeptide Y, peptide YY, pancreatic polypeptide, orexin A, orexin B, gastrin releasing peptide, bombensin, litorin, neuromedin B, neuromedin C, endothelin-1, endothelin-3, SDF-1, GROα, IL-8, melanocortin peptides, angiotensin II, bradykinin, cholestocytokinin, neuropeptide FF, and RFamide related peptides.

7. The method of claim 5, wherein the artificially modified ligand is a modified peptide ligand of the neuropeptide Y1 receptor.

8. The method of claim 2, further comprising releasing the drug molecule from the peptide ligand or protein ligand.

9. The method of claim 8, wherein release is accomplished by means of cleaving the cleavable linker moiety.

10. The method of claim 1, wherein the G-protein coupled receptor is selected from the group consisting of the neuropeptide Y1, Y2, Y4 or Y5 receptor, gastrin releasing peptide receptor, neuromedin B receptor, orexin receptor 1 or 2, bradykinin receptor 1 or 2, melanocortin receptor 1, 2, 3 or 4, CXCR2 or CXCR4 receptor, endothelin receptor A or B, angiotensin II receptor, cholecystokinin receptor 1 or 2, and neuropeptide FF receptor 1 or 2.

11. The method of claim 1, further comprising determining the internalization rate of the activated G-protein coupled receptor by using a fluorescently labeled G-protein coupled receptor and/or a fluorescently labeled peptide ligand or protein ligand, and particularly wherein the determination of the internalization rate is accomplished by means of fluorescence microscopy, fluorescence spectroscopy or an ELISA assay.

12. The method of claim 1, further comprising determining the internalization rate of the activated G-protein coupled receptor by using a radiolabeled G-protein coupled receptor and/or a radiolabeled peptide ligand or protein ligand, and particularly wherein the determination of the internalization rate is accomplished by means of scintillation counting of the radiolabel.

13. The method of claim 1, wherein the activated G-protein coupled receptor is internalized to the endosomes and/or lysosomes of the one or more target cells.

14. The method of claim 13, wherein the determination of the internalization rate of the activated G-protein coupled receptor further comprises the co-localization of the G-protein coupled receptor and/or the peptide ligand or protein ligand with lysosomal or late endosomal markers, and particularly wherein the lysosomal or late endosomal markers are selected from the group consisting of Rab7, Rab9, mannose-6-phosphate receptor, Lamp1, and Lamp2.

15. The method of claim 13, wherein the drug molecule is released from the peptide ligand or protein ligand intracellularly.
